(19) United States
(12) Patent Application Publication
(10) Pub. No.: US 2022/0013206 A1
(43) Pub. Date: Jan. 13, 2022

(54) SYSTEMS AND METHODS FOR EFFICIENT AND ECONOMICAL FULFILMENT OF PRESCRIPTION ORDERS

(71) Applicant: Slick Rx, LLC, West Bloomfield, MI (US)

(72) Inventors: Raad Shamoon Kouza, Van Buren Township, MI (US); Ramis Shamoon Kouza, Farmington Hills, MI (US); Anthony Ryiadh Asmar, West Bloomfield, MI (US)

(73) Assignee: Slick Rx, LLC, West Bloomfield, MI (US)

(21) Appl. No.: 17/370,806

(22) Filed: Jul. 8, 2021

Related U.S. Application Data
(60) Provisional application No. 63/049,454, filed on Jul. 8, 2020.

(51) Int. Cl. G16H 20/10; G16H 80/00; G06Q 30/02

(52) U.S. Cl. CPC G16H 20/10; G06Q 30/0283; G06Q 30/0282; G16H 80/00

(57) ABSTRACT

Disclosed are systems, methods, and storage media for operating a prescription-matching platform. Exemplary implementation may: receive, from a patient or a physician, an order for a medical supply where the order has a trait and is associated with the patient; select, from a network of pharmacies, a first list of network pharmacies meeting a trait of the order; select, from the first list of network pharmacies, a second list of pharmacies; communicate, to each pharmacy in the second list, a request to fill the order; select, by a pharmacy in the second list, the availability to fill the order; communicate, to the patient, the availability of each pharmacy in the second list the availability to fill the order; and select, by the patient, a pharmacy to fill the order.

### CROSS-REFERENCE TO RELATED APPLICATIONS

The present application claims priority to U.S. Provisional Patent Application No. 63/049,454, which was filed on Jul. 8, 2020 and is entitled Systems and Methods for Efficient and Economical Fulfilment of Prescription Orders.

### TECHNICAL FIELD

This disclosure relates generally to technological improvements in the fields of medical supply procurement, patient-pharmacy, patient-physician, and pharmacy-pharmacy transactions or interactions. More specifically, the present disclosure provides improved systems and methods for the procurement of prescription treatments, pharmaceuticals and other medical supplies, for patients.

### BACKGROUND

According to some estimates, prescription drug cost in the United States, account for 10% of overall health care spending and totals approximately $328 Billion annually—notice should be taken that this spending is at the consumer level, i.e., those who purchase the medications from pharmacies. Pharmacy Benefit Managers (PBMs) where part of an initial effort by insurers to lower the cost of prescription pharmaceuticals. In fact, when PBMs first began they served to reduce the cost of prescription pharmaceuticals by acting as the per-se middleman for insures by facilitating the negotiation and mass purchase of pharmaceuticals from pharmaceutical manufactures. In turn, PBMs where initially able to reduce the price of prescription drugs for the insurer and insured.

As often happens over time, however, incremental increase in the PBM's “fees” (among other cost) has led to a staggering increase in prescription and/or pharmaceutical cost. Of course, insurers cannot front the full increase in the increased cost and is passing the cost on to consumers—amount to the $328 Billion cost to the insured (e.g., tiers of covered drugs in an insurance plan that often results in higher co-pays or no coverage for new or quasi-experimental pharmaceuticals).

Today, independent pharmacies, generally independently owned and comprise of about 33% of all pharmacies, generate gross profits of less than about $18 billion. Whereas, the PBMs receive approximately $420 Billion in net profits—notably the net profits of the PBMs is higher than the TOTAL $328 Billion spent by the insured on the same products. Essentially, this equates to a credit card company making more money from the sale of a good by a local business per product (e.g., for an everyday household item, the credit card company makes a larger profit than the business selling the product). Accordingly, PBMs no longer benefit the insurer or the insured and, in fact, are driving up the cost of pharmaceuticals. Accordingly, there is a need for a more efficient, cost effective, mechanism to facilitate the exchange and delivery of pharmaceuticals and medical supplies, prescription, or otherwise, to the patient.

### SUMMARY OF THE INVENTION

In general, the present disclosure provides systems, methods, and storage media for operating a prescription-matching platform providing a cost effective mechanism that facilitates the exchange and delivery of pharmaceuticals to the patient. One aspect of the present disclosure relates to a method for dispensing a medical supply to a patient. The method may comprise of receiving, from a patient or a physician, an order for a medical supply where the order has a trait and is associated with the patient. The method may comprise of selecting, from a network of pharmacies, a first list of network pharmacies meeting a trait of the order. The method may comprise of selecting, from the first list of network pharmacies, a second list of pharmacies and communicating, to each pharmacy in the second list, a request to fill the order. The method may comprise of selecting, by a pharmacy in the second list, the availability to fill the order and communicating, to the patient, the availability of each pharmacy in the second list the availability to fill the order. The method may comprise of selecting, by the patient, a pharmacy to fill the order.

One aspect of the present disclosure relates to a system that facilitates the dispense of a medical supply to a patient. The system may comprise of hardware having one or more memories and processors. The memory (or memories) may store machine-readable instructions and the processor (or processors) may be configured to communicate with the memory to execute the machine-readable instruction. The processor may be configured to (e.g., the processor may be configured to execute the machine-readable instruction to) communicate with a physician electronic device that is associated with a physician and/or a patient electronic device that is associated with a patient. The processor may be configured to communicate with a pharmacy electronic device that is associated with a pharmacy, wherein the pharmacy is associated with a network of pharmacies. The processor may be configured to receive, from one of the physician electronic device or patient electronic device, an order for the medical supply where the order has a trait and is associated with the patient. The processor may be configured to select, from the network of pharmacies, a first list of network pharmacies meeting a trait of the order and to transmit, to the patient electronic device, the first list of network pharmacies. The processor may be configured to receive, from the patient electronic device, an indication representing a selection (e.g., patient makes a selection using the patient electronic device), from the first list of network pharmacies, of a second list of network pharmacies. The processor may be configured to receive, from each pharmacy electronic device of each pharmacy in the second list, an electronic indication of the availability to fill the order. The processor may be configured to transmit, to the patient electronic device, the availability of the pharmacies in the second list to fill the order. The processor may be configured to receive, from the patient electronic device, an indication representing a selection, from the second list of pharmacies and by the patient, of a selected pharmacy to fill the order. The processor may be configured to transmit, to the pharmacy electronic device of the selected pharmacy, the order to be filled by the selected pharmacy.

In some implementations of the system, the processor may be configured to generate a prescription ID associated with the order. The processor may be configured to transmit, to the patient electronic device, the prescription ID. The processor may be configured to transmit, to the pharmacy electronic device of the selected pharmacy, an option to confirm the prescription ID. The processor may be configured to receive, from the pharmacy electronic device, an input representative of the prescription ID. The processor may be configured to confirm the input, representative of the prescription ID, matches the prescription ID.

Another aspect of the present disclosure relates to a system that facilitates the dispense of a medical supply. The system may comprise hardware having one or more memories and processors. The memory (or memories) may store machine-readable instructions and the processor (or processors) may be configured to communicate with the memory to execute the machine-readable instruction. The processor may be configured to (e.g., the processor may be configured to execute the machine-readable instruction to) communicate with a plurality of pharmacy electronic devices, wherein each pharmacy electronic device is associated with a pharmacy within a network of pharmacies. The processor may be configured to receive, from a first one of the plurality of pharmacy electronic devices associated with a first pharmacy in the network of pharmacies, an indication identifying a need for the medical supply. The processor may be configured to transmit, to at least one of the pharmacy electronic devices of the remainder pharmacies in the network of pharmacies, a notification of the need for the medical supply. The processor may be configured to receive, from a second one of the plurality of pharmacy electronic devices associated with a second pharmacy in the network of pharmacies, an indication of the capability to transfer (e.g., sell or buy) the medical supply. The processor may be configured to transmit, to the first one of the plurality of pharmacy electronic device, an indication of the capability of the second pharmacy to transfer the medical supply to the first pharmacy. The processor may be configured to transmit a payment from the first pharmacy to the second pharmacy.

Other technical features may be readily apparent to one skilled in the art from the following figures, descriptions, and claims. These and other features, and attributes of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The present disclosure is best understood when read in conjunction with the accompanying drawings. It is to be expressly understood, however, that the drawings are for the purpose of illustration and description only and are not intended as a definition of the limits of the invention.

FIG. 1 generally illustrates a schematic diagram of a system of the present disclosure.

FIG. 2 generally illustrates a diagram of a method of the present disclosure.

### DETAILED DESCRIPTION

The present disclosure provides systems, methods, and storage media for operating a prescription-matching platform that provides a cost effective mechanism to facilitate the exchange and delivery of medical supplies to a patient. When compared to the systems and methods provided by PBMs, the presently disclosed systems and methods reduce the out-of-pocket (or patient) cost, while also increasing access to, medical supplies such as pharmaceuticals. Furthermore, the systems and methods of the present disclosure provides direct access to (i.e., makes available) the cost of a medical supply at any time (e.g., while the patient is with a physician). Thereby, the systems and methods of the present disclosure enhance the patient-physician relationship by allowing an accurate cost of a medical supply to be a factor in determining a course of treatment. Of course, one of skill in the art will readily appreciate many other advantages provided by the present disclosure.

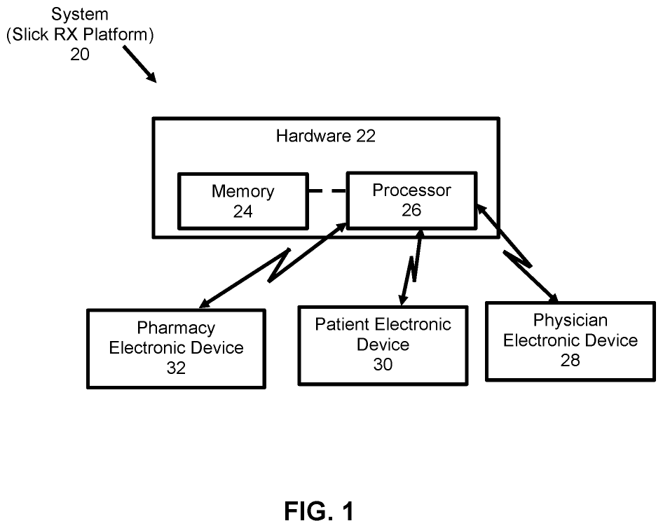

Referring now to FIG. 1, the present disclosure provides an exemplary system 20 that facilitates dispense of a medical supply to a patient. Herein, the system 20 may be referred to as and used interchangeably with “the RX Platform.” The RX Platform 20 comprises hardware 22 with one or more memories 24 and processors 26 in communication with one another (e.g., in electrical communication). The memory 24 (or memories) may store machine-readable instructions and the processor 26 (or processors) may be configured to communicate with the memory to execute the machine-readable instruction. In some embodiments, the RX Platform 20 may support an application (“RX App.”) (e.g., cloud software program, phone app or any other computer implemented network) with members and non-members. The members can include a network of patients, physicians and/or pharmacies. The application of the RX Platform 20 may also support non-members. For example, a non-member physician may write a prescription to a member patient—described further below.

The hardware 22 can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. Processor 26 may encompass any of the foregoing hardware, either singly or in combination. The memory 24 may comprise a single disk or a plurality of disks (e.g., hard drives), and includes a storage management module that manages one or more partitions within the memory 24. In some embodiments, memory 24 may include flash memory, semiconductor (solid state) memory or the like. The memory 24 may include Random Access Memory (RAM), a Read-Only Memory (ROM), or a combination thereof.

In some embodiments, the RX Platform 20 may comprise a physician electronic device 28 that is associated with a physician. The physician may be a member of the RX Platform 20 or may be a non-member. The physician may be any individual that may prescribe a prescription medication or any supplement to treat a condition of the patient. For example, the physician may be a medical doctor, hold an advance degree, certification, or have participated in specialized training to authorize the individual to write a prescription or recommend a supplement to treat a condition of the patient. The processors 26 may be configured to communicate with the physician electronic device 28. The processor 26 may electrically communicate with the physician electronic device 28 to facilitate communication between the RX Platform 20 and the physician electronic device 28 and in turn the physician. It is to be appreciate the physician electronic device 28 may be a computer, personal electronic device such as tablet, phone, watch, or any other electronic device (portable or non-portable, and including future electronic device). In some embodiments, the RX App may be downloaded to the physician electronic device 28 to facilitate communication between the RX Platform 20 and the physician electronic device 28 and physician.

In some embodiments, the RX Platform 20 may comprise a patient electronic device 30 that is associated with a patient. The patient may be a member of the RX Platform 20. If the patient is a non-member, the patient may use the patient electronic device 30 (e.g., via the RX App.) to set-up to become a member of the RX Platform 20. The processors 26 may be configured to communicate with the patient electronic device 30. The processor 26 may be configured to be electrically communicate with the patient electronic device 30 to facilitate communication between the RX Platform 20 and the patient electronic device 30 and in turn the patient. It is to be appreciate that the patient electronic device 30 may be a computer, personal electronic device such as a tablet, phone, watch, or any other electronic device (portable or non-portable, and including future electronic device). In some embodiments, a RX App may be downloaded to the patient electronic device 30 to facilitate communication between the RX Platform 20 and the patient electronic device 30 and patient.

In some embodiments, the RX Platform 20 may facilitate a telehealth or other virtual healthcare platform or application. Accordingly, the physician and patient may use the Slix RX App—via respective patient and physician electronic devices 28, 30—to initiate and participate in a telehealth or other virtual healthcare appointment. In some embodiments, the RX Platform 20 and Slix RX App may be used independent of the forum of meeting between the patient and the physician. For example, the patient and physician may meet in an office or virtually, and the RX App may not be used until after the meeting. In another example, the RX Platform 20 may host a virtual healthcare platform and the RX App, thereby facilitating use of the RX App during the patient-physician consultation.

In some embodiments, the RX Platform 20 may confirm a patient-physician relationship. The confirmation could occur regardless of the forum (e.g., telehealth or in a physical office) for meeting between the physician and patient. In some embodiments, the processor 20 may be configured to send a patient ID to the patient through the patient electronic device 30. Contemporaneously, or at any time, the processor 26 may be configured to send a notice to the physician through the physician electronic device 28 by a pop-up in the physician electronic device 28. The physician than may use the physician electronic device 28 to enter a patient ID that would be/is provided by the patient. The processor may also be configured to receive the patient ID and determine if it matches the patient code sent to the patient. If it does, the processor 26 confirms a patient-physician relationship between a patient and a physician.

In some embodiments, the RX Platform 20 may comprise a pharmacy electronic device 32 that is associated with a pharmacy. The pharmacy may be a member of the RX Platform 20 and associated with a network of pharmacies. The processors 26 may be configured to communicate with the pharmacy electronic device 32. The processor 26 may be configured to electrically communicate with the pharmacy electronic device 32 to facilitate communication between the RX Platform 20 and the pharmacy electronic device 32 and in turn the pharmacy. It is to be appreciate that the pharmacy electronic device 32 may be a computer, personal electronic device such as tablet, phone, watch, or any other electronic device (portable or non-portable, and including future electronic device). In some embodiments, the RX App may be downloaded to the pharmacy electronic device 32 to facilitate communication between the RX Platform 20 and the pharmacy electronic device 32 and pharmacy.

The processor 26 may be configured to receive, from one of the physician electronic device 28 and the patient electronic device 30, an order for a medical supply. The medical supply may be a prescription or non-prescription (e.g., over-the-counter) pharmaceutical, a durable medical device, or any other type of medical supply. In some embodiments, the medical supply may be a prescription pharmaceutical or medication. Although primarily referred to herein as a prescription pharmaceutical, and as noted above, the medical supply is not to be limited. In some embodiments, when a patient-physician relationship has been confirmed, the processor 26 may be configured to receive, from the physician electronic device 28, the order of a prescription that is associated with a patient. In some embodiments, the patient may use the patient electronic device 30 to submit the order using a physical prescription received from a non-member physician. For example, the patient may manually input the details of the physical prescription into the RX App on the patient electronic device 30. In one embodiment, the patient may be able to use the RX App and patient electronic device to upload an image of the physical prescription (e.g., by using a camera or scanner of the patient electronic device.). In such an instance, the processor 26 may be configured to receive the prescription submission.

In some embodiments, the order may be associated with a patient that has a trait. In some embodiments, the trait may be a location of the patient such as the patient's home address. In some embodiments, the processor 26 may be configured to receive a physical location (e.g., GPS location) of the patient electronic device 30. The processor 26 may associated the physical location of the patient electronic device 30 with a physical location of the patient and associate (or identify) the physical location as a trait. It is to be appreciated that the physical location may be based on a current or past dynamic (e.g., moving) and/or static physical location of the patient electronic device 30 or the patient. In some embodiments, the trait of the order may be a time the medical supply may be available to the patient. In some embodiments, the processor 26 may be configured to calculate the approximate time the medical supply may be available for pick-up by the patient, where the pick-up time is based, at least in-part, on the travel time from the patient's physical location to a pharmacy.

In some embodiments, the trait may also be a price of the prescription order. The price of the prescription order may be a set price in the Slick RX Platform or the price may be dynamic (e.g., change at an interval based on supply and demand of a certain medical supply). In some embodiments, the Slick RX System 20 sets a price for the medical supply based on a pre-negotiated arrangement with the medical supply manufacturer. In some embodiments, the processor 26 may be configured to transmit to the patient electronic device 30 and/or the physician electronic 28 the price of the medical supply. The patient and/or physician may use the price of the medical supply in determining a treatment. For example, if the medical supply is a prescription pharmaceutical, one prescription pharmaceutical may be less expensive over another, and the physician could utilize the price as one factor in advising the patient on a treatment.

In some embodiments, the processor 26 may be configured to receive, from the patient electronic device 30, a selection, by the patient, the trait of the order. For example, the patient may select that the medical supply be located less than 10 minutes from the patient's home. In some embodiments, the trait may be the option to pick-up or have the medical supply delivered. Accordingly, the patient could select to view only those medical supplies that may be delivered or picked-up. Of course, one of skill in the art will appreciate the multitude of variables or characteristics the trait may be or have, and that may be selected by the patient.

In some embodiments, the processor 26 may be configured to select, from the network of pharmacies, a first list of network pharmacies meeting a trait of the order. The first list of network pharmacies may be selected based on a physical location of the patient, availability of the medical supply, or any number of other traits associated with the order. In some embodiments, the processor 26 may be configured to transmit, to the patient electronic device 30, the first list of network pharmacies. For example, the processor 26 may send to the patient electronic device 30 a listing of network pharmacies that are within a pre-defined geographical location or that have a medical supply in stock. Once received, the patient electronic device 30 may display the first list of network pharmacies and prompt the patient to make a selection from the first list of network pharmacies. In some embodiments, the processor 26 may be configured to receive, from the patient electronic device 28, an indication representing a selection, from the first list of network pharmacies, of a second list of network pharmacies. For example, the patient may view the listing of network pharmacies and select one that is located closest to the patient's home and has the medical supply in stock. The second list of pharmacies may be one or more pharmacies. It is also contemplated the patient may request a pharmacy not in the first list (e.g., a pharmacy the patient normally uses, but based on the patients physical location or due to the medical supply being out of stock is not being displayed).

In some embodiments, the processors 26 may be configured to receive, from each pharmacy electronic device 32 of each pharmacy in the second list, an electronic indication of the availability to fill the order. For example, the processor 26 may receive an electronic indication associated with a network pharmacies' stock and ability of fill an order. In some embodiments, the processor 26 may transmit a notice to each pharmacy electronic device 32 of each pharmacy in the second list that the pharmacy has been selected to fill and order, and to request the pharmacy transmit a notice to the RX Platform 20, which is received by the processor 26, of the medical supplies availability. For example, the processor may 26 may notify, via an electronic notice (e.g., email, text, or other indication) that the pharmacy may fill an order, and the notice provide an option for the pharmacy to select if it will accept or decline fulfilling the order. In some embodiments, the availability of the medical supply at each network pharmacy is automatically updated and kept current in the memory 24 and/or automatically communicated to the processor 26.

In some embodiments, the processor 26 may be configured to transmit, to the patient electronic device 20, the availability of the pharmacies in the second list to fill the order. For example, the processor 26 may transmit, to the patient electronic device 20, that certain network pharmacies are capable of filling the order. The processor 26 may be configured to receive, from the patient electronic device 30, an indication representing a selection by the patient of a selected pharmacy, from the second list of pharmacies, to fill the order. For example, the processors 26 may receive a notice that the patient selected a certain network pharmacy from the second list. In some embodiments, the processor 26 may be configured to transmit, to the pharmacy electronic device 32 of the selected pharmacy, that the order is to be filled by the selected pharmacy. In other words, the RX Platform 20 communicates with each of the patient and pharmacy that the order is to be filled and picked-up at a selected network pharmacy.

In some embodiments, the processor 26 may be configured to generate a prescription ID associated with the order. The prescription ID may be used by the patient and pharmacy, similar to the patient ID described above, to confirm the order and identity of the patient prior to the pharmacy providing the patient with the order. In some embodiments, the processor may be configured to transmit, to the patient electronic device 30, the prescription ID. In some embodiments, the patient electronic device 30 is configured to transmit, to the pharmacy electronic device 32 of the selected pharmacy, an option to confirm the prescription ID. With the option in the pharmacy electronic device 32 available to the pharmacy, the patient may provide the prescription ID received by the patient to the pharmacy. In some embodiments, the processor is configured to receive, from the pharmacy electronic device 32, an input representative of the prescription ID (e.g., pharmacy enters the prescription ID provided by the patient). In some embodiments, the processor may be configured to confirm that the input, representative of the prescription ID, matches the prescription ID.

In some embodiments, the RX Platform 20, through the patient electronic device 30 or the pharmacy electronic device 32, may provide payment for the order. In some embodiments, the processors 26 may be configured to receive a notice of payment, representative of a payment received by the pharmacy from the patient, from the pharmacy electronic device 32. In other words, a patient may provide cash or otherwise make a payment to a pharmacy, and the pharmacy may provide a credit to an account associated with the patient in the RX Platform 20. In some embodiments, payment for the order may be made through the RX Platform 20 using the patient electronic device 30 or pharmacy electronic device 32 (e.g., patient has a credit on his account and the pharmacy request a debit from the patients account). In some embodiments, the patient may pay the pharmacy directly (e.g., with cash or credit card) for the order.

In some embodiments, the processor 26 may be configured to communicate with a plurality of pharmacy electronic devices, wherein each pharmacy electronic device is associated with a pharmacy within a network of pharmacies (e.g., provide communication between the network of pharmacies). In some embodiments, the processor 26 may be configured to receive, from a first one of the plurality of pharmacy electronic devices associated with a first pharmacy in the network of pharmacies, an indication identifying a need for the medical supply. For example, one network pharmacy may desire to fill an order but does not have the medical supply in stock. Accordingly, the network pharmacy may send a notice to other network pharmacies, such as near-by, pharmacies to obtain the medical supply. In some embodiments, the processors 26 may be configured to transmit, to the pharmacy electronic devices of the remainder pharmacies in the network of pharmacies, a notification of the need for the medical supply (e.g., the RX Platform 20, via the processors 26 and pharmacy electronic devices 32, notifies the network of pharmacies of the need for the medical supply). The need may be a request for (e.g., to purchase) or to sell excess of a medical supply. For example, a pharmacy may input into the RX App its need for or to sell a medical supply. Once input into the RX App and received by the processor 26, the processor 26 may sends a notice to the pharmacy electronic device 32 of the remainder pharmacies in the network of the need. In some embodiments, no notice is provided, but the processor 26 is configured to display a listing of a need associated with a network pharmacy. In some embodiments, the processor 26 is configured to receive, from a second one of the plurality of pharmacy electronic devices associated with a second pharmacy in the network of pharmacies, an indication of the capability to transfer the medical supply (e.g., a network pharmacy has excess stock of a medical supply and can sell it to the pharmacy in need). For example, a network pharmacy may receive the notice of the need for a medical supply and desire to sell the medical supply. In some embodiments, the processors is configured to transmit, to the first one of the plurality of pharmacy electronic device, an indication of the capability of the second pharmacy to sell the medical supply to the first pharmacy. In some embodiments, the processor 26 is configured to transmit a payment from the first pharmacy to the second pharmacy (e.g., through the RX Platform).

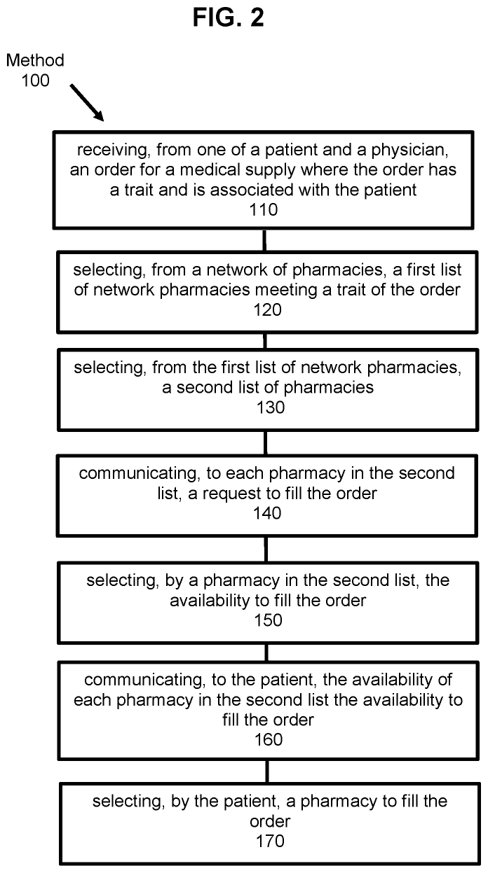

Referring to FIG. 2, the present invention provides a method 100 for dispensing a medical supply to a patient. It is to be appreciated the following description of the method may be employed by the systems disclosed above, or in any equivalent or similar system or apparatus. It should also be appreciated, in view of the above disclosure, that the foregoing method may be implemented by, and using, the RX Platform 20. The method may comprise the step 110 of receiving, from one of a patient and a physician, an order for a medical supply where the order has a trait and is associated with the patient. In some embodiments of the method, the order is an order for a prescription medication. The method may comprise the step 120 of selecting, from a network of pharmacies, a first list of network pharmacies meeting a trait of the order. In some embodiments of the method, the trait of the order is based on a location of the patient. In some embodiments of the method, the location is a physical location of the patient, and the physical location may be a current or dynamic location of the patient. In some embodiments, the trait may be a price of the prescription order.

The method may comprise step 130 of selecting, from the first list of network pharmacies, a second list of pharmacies. The method may comprise the step 140 of communicating, to each pharmacy in the second list, a request to fill the order. The method may comprise the step 150 of selecting, by a pharmacy in the second list, the availability to fill the order. The method may comprise the step 160 of communicating, to the patient, the availability of each pharmacy in the second list the availability to fill the order. The method may comprise the step 170 of selecting, by the patient, a pharmacy to fill the order.

Some embodiments of the method may comprise the step of generating a prescription ID associated with the order. Some embodiments of the method may comprise the step of communicating, to the patient, the prescription ID. Some embodiments of the method may comprise the step of communicating, to the pharmacy, an option to confirm the prescription ID. Some embodiments of the method may comprise the step of receiving, by the pharmacy from the patient, an indication of the prescription ID. Some embodiments of the method may comprise the step of communicating, by the pharmacy, the indication of the prescription ID. In Some embodiments of the method may comprise the step of communicating one of a rejection and confirmation that the indication of the prescription ID matches the prescription ID.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The word “example” is used herein to mean serving as an example, instance, or illustration. Any aspect or design described herein as “example” is not necessarily to be construed as preferred or advantageous over other aspects or designs. Rather, use of the word “example” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment or implementation unless described as such.

Implementations of the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are available.

The above-described embodiments, implementations, and aspects have been described in order to allow easy understanding of the present invention and do not limit the present invention. On the contrary, the invention is intended to cover various modifications and equivalent arrangements included within the scope of the appended claims, which scope is to be accorded the broadest interpretation to encompass all such modifications and equivalent structure as is permitted under the law.

What is claimed is:

1. A system facilitating dispense of a medical supply to a patient, the system comprising:
a memory for storing machine-readable instructions;
a processors in communication with the memory wherein the processor is configured to execute the machine-readable instructions to:
communicate with a physician electronic device associated with a physician;
communicate with a patient electronic device associated with a patient;
communicate with a pharmacy electronic device associated with a pharmacy, wherein the pharmacy is associated with a network of pharmacies;
receive, from one of the physician electronic device and patient electronic device, an order for the medical supply where the order has a trait and is associated with the patient;
select, from the network of pharmacies, a first list of network pharmacies meeting a trait of the order;
transmit, to the patient electronic device, the first list of network pharmacies;
receive, from the patient electronic device, an indication representing a selection, from the first list of network pharmacies, of a second list of network pharmacies;
receive, from each pharmacy electronic device of each pharmacy in the second list, an electronic indication of the availability to fill the order;
transmit, to the patient electronic device, the availability of the pharmacies in the second list to fill the order;
receive, from the patient electronic device, an indication representing a selection by the patient of a selected pharmacy, from the second list of pharmacies, to fill the order; and
transmit, to the pharmacy electronic device of the selected pharmacy, the order to be filled by the selected pharmacy.

2. The system of claim 1, wherein the order is as an order for a prescription medication.

3. The system of claim 1, wherein the trait of the order is based on a location received from the patient electronic device.

4. The system of claim 3, wherein the location received from the patient electronic device is based on a physical location of the patient electronic device.

5. The system of claim 1, wherein the processor is configured to receive, from the patient electronic device, a selection by the patient of the trait of the order.

6. The system of claim 5, wherein the trait is one of picking-up the order and having the order delivered.

7. The system of claim 1, wherein the trait of the order is a price of the medical supply.

8. The system of claim 7, wherein the processor is configured to transmit, to one of the physician electronic device and the patient electronic device, a price of the order.

9. The system of claim 2, wherein the processor is further configured to:
generate a prescription ID associated with the order;
transmit, to the patient electronic device, the prescription ID;
transmit, to the pharmacy electronic device of the selected pharmacy, an option to confirm the prescription ID;
receive, from the pharmacy electronic device, an input representative of the prescription ID;
confirm the input, representative of the prescription ID, matches the prescription ID.

10. The system of claim 1, wherein the trait of the order is a time the medical supply may be available to the patient.

11. The system of claim 1, wherein one of the physician and patient electronic device is further defined as personal electronic device.

12. The system of claim 1, wherein the processor is further configured to receive a notice of payment, representative of a payment received by the pharmacy from the patient, from the pharmacy electronic device.

13. The system of claim 12, wherein the notice of payment is credited to an account associated with a patient.

14. A system facilitating dispense of a medical supply, the system comprising:
a memory for storing machine-readable instructions;
a processors in communication with the memory wherein the processor is configured to execute the machine-readable instructions to:
communicate with a plurality of pharmacy electronic devices, wherein each pharmacy electronic device is associated with a pharmacy within a network of pharmacies;
receive, from a first one of the plurality of pharmacy electronic devices associated with a first pharmacy in the network of pharmacies, an indication identifying a need for the medical supply;
transmit, to at least one of the pharmacy electronic devices of the remainder pharmacies in the network of pharmacies, a notification of the need for the medical supply;
receive, from a second one of the plurality of pharmacy electronic devices associated with a second pharmacy in the network of pharmacies, an indication of the capability to transfer the medical supply;
transmit, to the first one of the plurality of pharmacy electronic device, an indication of the capability of the second pharmacy to sell the medical supply to the first pharmacy; and
transmit a payment from the first pharmacy to the second pharmacy.

15. A method for dispensing a medical supply to a patient, the method comprising:
receiving, from one of a patient and a physician, an order for a medical supply where the order has a trait and is associated with the patient;
selecting, from a network of pharmacies, a first list of network pharmacies meeting a trait of the order;
selecting, from the first list of network pharmacies, a second list of pharmacies;
communicating, to each pharmacy in the second list, a request to fill the order;
selecting, by a pharmacy in the second list, the availability to fill the order;
communicating, to the patient, the availability of each pharmacy in the second list the availability to fill the order; and
selecting, by the patient, a pharmacy to fill the order.

16. The method of claim 15, wherein the order is as an order for a prescription medication.

17. The method of claim 15, wherein the trait of the order is based on a location of the patient.

18. The method of claim 17, wherein the location received from the patient electronic device is based on a physical location of the patient.

19. The system of claim 15, wherein the trait of the order is a price of the order.

20. The method of claim 16 further comprising:
generating a prescription ID associated with the order;
communicating, to the patient, the prescription ID;
communicating, to the pharmacy, an option to confirm the prescription ID;
receiving, by the pharmacy from the patient, an indication of the prescription ID;
communicating, by the pharmacy, the indication of the prescription ID; and,
communicating one of a rejection and confirmation that the indication of the prescription ID matches the prescription ID.
